(19) United States
(12) Patent Application Publication
(10) Pub. No.: US 2010/0191089 A1
(43) Pub. Date: Jul. 29, 2010

(54) METHOD AND SYSTEM TO DETECT NEOINTIMA COVERAGE OF A STENT

(75) Inventors: Kathrina Stebler, Uster (CH); Nils Kucher, Uster (CH)

Correspondence Address: BIRCH STEWART KOLASCH & BIRCH, PO BOX 747, FALLS CHURCH, VA, 22040-0747, US

(73) Assignee: Sis Medical AG, Winterthur (CH)

(21) Appl. No.: 12/668,319

(22) PCT Filed: Jul. 9, 2008
(86) PCT No.: PCT/CH08/00306

§ 371 Date: Mar. 4, 2010

Related U.S. Application Data
(60) Provisional application No. 60/958,838, filed on Jul. 9, 2007.
(60) Provisional application No. 60/958,878, filed on Jul. 10, 2007.

(51) Int. Cl. A61B 5/053

(52) U.S. Cl. 600/373; 600/547

(57) ABSTRACT

A method and endovascular system, such as a balloon catheter, for detecting the absence of neointimal coverage of bare or drug-eluting metal stents, featuring two (bipolar systems) or more electrodes (multipolar systems). The distal part of the device has an expandable platform, such as an inflatable balloon, for providing transient mechanical contact of the electrodes to the stented vessel segment. The electrodes on the expandable platform have a circumferential-symmetric array, i.e., arrangement in identical circular sectors. For example, the electrodes are arranged in semicircles, i.e., at 90 and 180 degrees, when using a bipolar catheter system, or they are arranged in quarter-sections, i.e., at 90, 180, 270, and 360 degrees, when using a quadrupolar catheter system. The electrodes of the endovascular device connect with a direct current (DC) or alternating current (AC) measurement unit. With multipolar systems, rotational impedance measurement technology is used for accurate detection of exposed (non-covered) stent struts and is defined herein as sequential (clock-wise or counterclock-wise) testing of each single electrode against the remaining electrically interconnected electrodes. The described endovascular system requires direct mechanical contact of at least 2 electrically non-interconnected electrodes with the stent to induce short-circuit current. The method and system can distinguish complete neointimal stent coverage, defined as consistently high impedance values in all measurements, from partial neointimal coverage, defined as a mix of high and low impedance values, from missing neointimal coverage, defined as consistently low impedance values in all measurements.

Percutaneous coronary intervention (PCI) e.g. using balloon angioplasty and implantation of stents is performed worldwide in millions of patients with coronary artery disease each year. For many years, balloon angioplasty without stent implantation was used to treat coronary obstructions. Due to the risk of restenosis of approximately 30-40% from vessel recoil and excessive neointimal proliferation, bare metal stents have been introduced to reduce the risk of restenosis to approximately 20%. The risk of restenosis was reduced to less than 10% with the introduction of first-generation drug-eluting stents in 2002. The only two drug-eluting stents that have been approved yet by the U.S. Food and Drug Administration are the Cypher® stent (Cordis) and the Taxus® stent (Boston Scientific). These stents are covered with a polymer that slowly releases active drug (sirolimus in Cypher®, paclitaxel in Taxus® stents) that is known to inhibite neointimal proliferation. The introduction of these devices has substantially reduced the need for target lesion revascularization from instent restenosis.

Inhibition of neointimal proliferation by drug-eluting stents may on the one hand reduce the restenosis rate but on the other hand may disturbe endothelialization of stents. A thin layer of intima appears to be important to prevent stent thrombosis. The main shortcoming of first-generation drug-eluting stents is the continued risk of late stent thrombosis that was rarely observed with bare metal stents. Recent evidence confirmed that there is an approximately 0.6% incidence per year in the rate of late stent thrombosis(1). Unfortunately, this risk persists even several years after stent implantation, suggesting that endothelialization may not only be delayed but completely inhibited. Stent thrombosis is life-threatening with a mortality rate of up to 45%(2). In the randomized controlled trials of the Cypher and Taxus stents, there is an increase in the rate of late stent thrombosis as compared with the use of bare metal stents(3). These trials, however, were not powered to detect a difference in the death rate between the drug-eluting stent and the bare metal stent patients. In the recent large Swedish registry of almost 20000 patients, drug-eluting stents were associated with a significant increase in the mortality rate as compared with bare metal stents(4). Despite the risk of reduced endothelialization and stent thrombosis, drug-eluting stents are still being implanted in many patients with coronary artery disease due to the great benefit in preventing restenosis.

Several facts support the hypothesis that missing or incomplete neointimal stent coverage is a trigger of late stent thrombosis: 1) premature discontinuation of dual antiplatelet therapy is an independent and powerful predictor of late stents thrombosis(2), 2) late incomplete stent apposition due to positive vessel wall remodeling occurs in up to 5.1% of the drug-eluting stent patients and appears to be a predictor of late stent thrombosis(5, 6), 3) delayed or missing endothelialization(7), and 4) hypersensitivity to or inflammation from the polymer.

There are 3 imaging techniques that have been used to detect incomplete neointimal coverage or incomplete stent apposition:

1) Intravascular ultrasound (IVUS)

2) Optical coherence tomography (OCT)

3) Coronary angioscopy

It has been shown that IVUS is not sufficient to detect stent endothelialization due to its low resolution(5). Although OCT is more sensitive than IVUS, its resolution may also be to low to detect thin endothelial layers. For example, in a recent study, OCT revealed that only 16% of the sirolimus-eluting stents had complete endothelial coverage(5). Other disadvantages of OCT are that ostial stents cannot be imaged because the proximal blood vessel must be transiently occluded by a blocking balloon and the blood removed by injection of saline before clear images are obtained. Many patients suffer chest pain with ST segment deviations from prolonged (more than 1 minute) coronary occlusion. Similarly, full visibility or translucency of stent struts by coronary angioscopy may not be sensitive enough to detect thin layers of neointima(7).

Electric impedance spectroscopy has been investigated for its suitability to detect thickness of present tissue like neointima on the surface of stents per se (11). However, upon confirmation of basic suitability of electric impedance spectroscopy for existing tissue, no feasible method to sufficiently examine the inner surface of implanted stents has been disclosed. Particularly, no method or system has been disclosed that enables the detection of uncovered stent areas, i.e., to the blood stream exposed stent struts without any endothelialization. The Süselbeck study shows four prototype micro-electrodes arranged at the surface of the balloon of a catheter in a short line of about 1 mm length and parallel to the longitudinal axis of the balloon.

Further electric impedance methods refer to the position of the balloon of the catheter relative to the stent only, as is shown by example in U.S. Pat. No. 5,749,914.

The detection of missing or incomplete neointimal coverage, of coronary stents is in need for the following reasons:
In patients with drug-eluting stent implants, the information that the stents are covered by neointima may be helpful for predicting the risk of future stent thrombosis events.
Patients with drug-eluting stent implants and documented complete stent coverage may not need prolonged dual antiplatelet therapy that increases the rate of bleeding complications(8-10).
Patients with drug-eluting stent implants and missing stent coverage may need prolonged or indefinite dual antiplatelet therapy to reduce the risk of late stent thrombosis.
In patients with drug-eluting stent implants who are candidates for premature discontinuation of dual antiplatelet therapy, for example, in case of malcompliance, side effects from clopidogrel or aspirin, bleeding complications, or need for non-cardiac surgery, the information of stent coverage may be important for planning the patients care.
Accurate information about neointimal coverage is of great importance for the development of new-generation drug-eluting stents with similar efficacy in preventing restenosis but with improved safety regarding the risk of late stent thrombosis.

It is therefore an object of the present invention to provide a method and a system to detect neointima coverage, or lack of neointima coverage respectively, of a stent implanted in a vessel.

Accordingly, a method is provided for a use of a sensor having a sensing section adapted to be introduced into a stent and including a plurality of electrodes arranged in a pattern circumferentially extending on the outer surface area of the sensing section, and being further arranged for operationally contacting the inner surface of the stent, whereby the electrodes are connected to a measuring unit adapted to measure DC resistance and/or AC impedance between preselected electrodes according to a measurement condition established during a measurement session, whereby the measurement condition is altered during a measurement session for consecutive measurements over various electrode combinations according to a preselected procedure, and in that measurement results with low DC resistance and/or low AC impedance with a small angular shift between voltage and current identify presence of areas of the inner surface of the stent with lacking coverage of neointima.

This method allows to make use of e.g. angioplastic balloons covered with a pattern of electrodes in the inventive manner, i.e. to perform electric impedance spectroscopy over the whole relevant inner surface of the stent in order to detect even small but relevant areas of the inner stent surface not covered with neointima. A circumferentially extending pattern of electrodes thereby permits to detect presence or absence of neointima over all the inner circumference of the stent and over the length of the sensing section, such that smaller areas of irregular neointima coverage can be detected wherever located on the inner surface of the stent. The distance between associated electrodes determines the dimension of the areas detectable.

Such an electrode is connectable to any kind of measurement unit as described below. Associated electrodes are electrodes connectable by the switching means according to a measurement condition for resistance or impedance measurement.

A further aspect of the invention provides for a sensor to detect the intima coverage of a stent with a sensing section, adapted to be introduced into a stent and including electrodes arranged on the outer surface area of the sensing section, and a shaft adapted to move the sensing section within a vessel of a body towards the position of a stent being implanted in the vessel and to move the sensing section operationally into this stent, the shaft being further adapted to accommodate conductors to connect the electrodes operationally with a measuring unit, whereby the electrodes are arranged for contacting the inner surface of the stent, and whereby the electrodes are further arranged such that the direction of a current flowing between associated electrodes is substantially transverse to the longitudinal axis of the sensing section.

Another aspect of the invention provides for a sensor to detect the intima coverage of a stent with a sensing section, adapted to be introduced into a stent and including electrodes arranged on the outer surface area of the sensing section, and a shaft adapted to move the sensing section within a vessel of a body towards the position of a stent being implanted in the vessel and to move the sensing section operationally into this stent, the shaft being further adapted to accommodate conductors to connect the electrodes operationally with a measuring unit, whereby the electrodes are arranged for contacting the inner surface of the stent, and whereby the sensing section comprises at least one electrode extending in a direction substantially parallel to the axis and over substantially the length of the sensing section.

Such arrangements of the electrodes, in particular according to claims 6 to 9, advantageously allow to simplify the pattern of electrodes arranged on the sensing section, e.g. a balloon, and therefore to minimize the number of conductors, i.e., leads as needed from the sensing section along the shaft to a measuring unit for supplying voltage and current to the electrodes and for analyzing electrode data for determining presence or absence of neointima. As the length of a stent is a multiple of its diameter, the length of electrodes extending lengthwise on the sensing section is increased compared to the length of annular electrodes located at the proximal and at the distal end of the sensing section; consequently the inner surface area contacted by the lengthwise extending electrodes is larger, thereby increasing the detected surface area, and a current flow due to a contact of the electrodes with the inner surface of the stent is directed substantially transverse to the longitudinal axis of the stent.

An other aspect of the invention provides for a measuring unit comprising coupling means to electrically connect the two conductors on a shaft of a sensor to detect the intima coverage of an implant, a DC generator, means adapted to connect the DC generator with the two conductors to establish measurement condition of a measurement session, and DC resistance measuring means adapted to measure DC resistance between conductors connected to the DC generator, and output means for transmitting and/or displaying measurement results.

Basic inventive intima detection is thereby possible with simple equipment for detection of areas with irregular intima coverage of comparably larger dimensions.

Another aspect of the invention provides for a measuring unit comprising coupling means to electrically connect the at least three conductors on a shaft of a sensor to detect the intima coverage of an implant, a DC generator, switching means adapted to connect the DC generator in a predetermined manner with the at least three conductors to establish measurement condition of a measurement session, and DC resistance measuring means adapted to measure DC resistance between conductors connected to the DC generator according to the measurement condition as established, and further being adapted for altering the measurement condition during the measurement session according to a predetermined procedure to perform resistance measurement over various electrode combinations, storage means for storing measurement results, output means for transmitting and/or displaying measurement results, and a control unit adapted to control the procedure of a measurement session.

The use and analysis of three or more electrodes allows to detect small areas with irregular intima coverage; the more electrodes are analyzed, the smaller are the areas detectable.

Another aspect of the invention provides for a measuring unit comprising coupling means to electrically connect the two conductors on a shaft of a sensor to detect the intima coverage of an implant, a AC generator, means adapted to connect the AC generator with the two conductors to establish measurement condition of a measurement session, and AC complex impedance measuring means adapted to measure AC impedance between the two conductors connected to the AC generator, and output means for transmitting and/or displaying measurement results.

By using AC instead of DC, adverse effects on the living body might be avoided, in particular if measurements need to be repeated.

Another aspect of the invention provides for a measuring unit comprising coupling means to electrically connect the at least three conductors on a shaft of a sensor to detect the intima coverage of an implant, an AC generator, switching means adapted to connect the AC generator in a predetermined manner with the at least three conductors to establish measurement condition of a measurement session, and AC complex impedance measuring means adapted to measure complex AC impedance between conductors connected to the AC generator according to the measurement condition as established, and further being adapted for altering the measurement condition during the measurement session according to a predetermined procedure to perform complex impedance measurement over various electrode combinations, storage means for storing measurement results, output means for transmitting and/or displaying measurement results, and a control unit adapted to control the procedure of a measurement session.

Another aspect of the invention provides for a measuring unit comprising coupling means to electrically connect the unit with the at least four conductors on a shaft of a sensor to detect the intima coverage of an implant, an AC generator, a complex impedance detecting arrangement, switching means adapted to connect two of the conductors with the AC generator and the remaining conductors with the complex impedance detecting arrangement to establish measurement condition, the switching means being further adapted for altering the measurement condition during the measurement session according to a predetermined procedure to perform impedance measurement over various electrode combinations, storage means for storing measurement results, output means for transmitting and/or displaying measurement results, and a control unit adapted to control the procedure of a measurement session.

In such a measuring unit, adequate bridging techniques in the complex impedance arrangement may be used for detecting impedance.

A measuring unit as described above allows to operatively connect the electrodes of the sensing section of a sensor in various combinations in order to detect any inner surface area of the stent not covered with neointima independent of its location relative to the electrodes. The measurement session includes to establish as much measurement conditions as needed to completely make use of all useful electrode combinations.

An other aspect of the invention includes a method to determine neointima coverage on the inner surface of a stent characterized in that a sensing section having a pattern of electrodes arranged on its outer surface is introduced into the stent such that the electrodes are in contact with the inner surface of the stent, and impedance measurements between selected electrodes are performed, whereby short circuits are distinguished from enhanced impedance between these electrodes and short circuits are interpreted as lack of intima coverage in the area of these electrodes.

In addition, this Method can be performed, whereby increased angular phase shift in the impedance measured is interpreted as increased thickness of intima coverage in the area of these electrodes.

Furthermore, this Method can be performed according to one of the preceeding steps, whereby the pattern of electrodes is arranged such to evenly extend over the surface of the sensing section and selection of particular electrodes for an impedance measurement is done such that various combinations of electrodes for a different measurement have occurred at least once.

It is well understood that the present invention can be used to detect neointima coverage or lack of coverage not only in stents, but in any kind of implant, provided that neointima coverage is an issue as described above.

Preferred embodiments are described in the dependent claims.

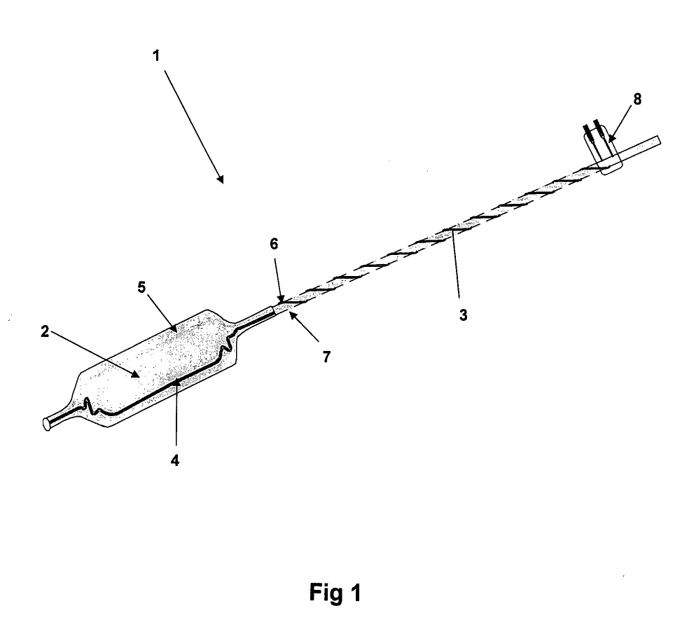

FIG. 1 shows a sensor according to the invention in a perspective view,

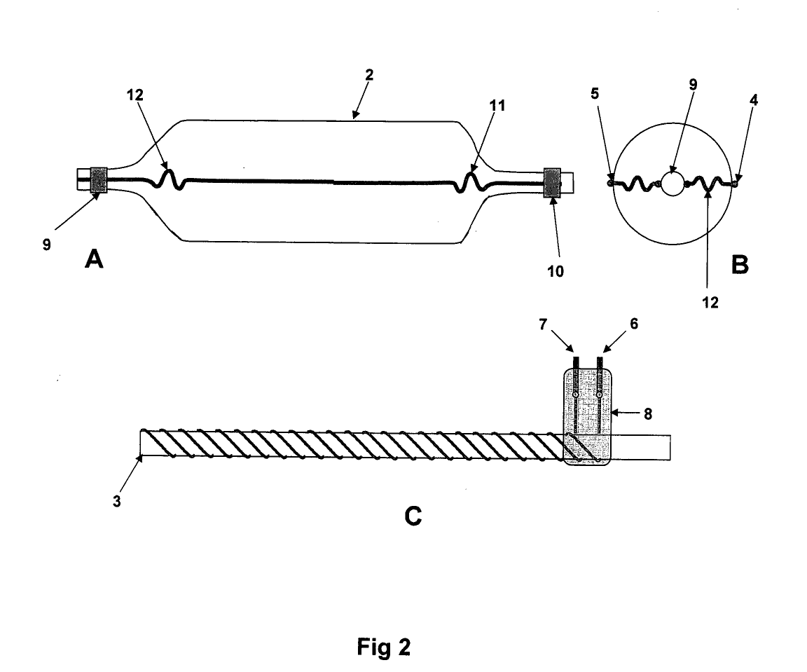

FIG. 2 shows the sensor of FIG. 1 in a different view,

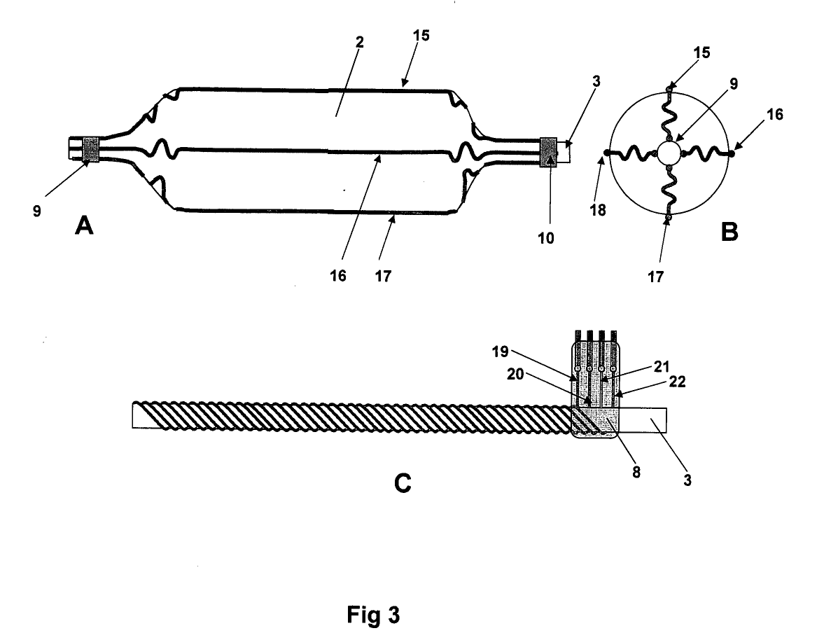

FIG. 3 shows a preferred embodiment of the sensor according to the invention,

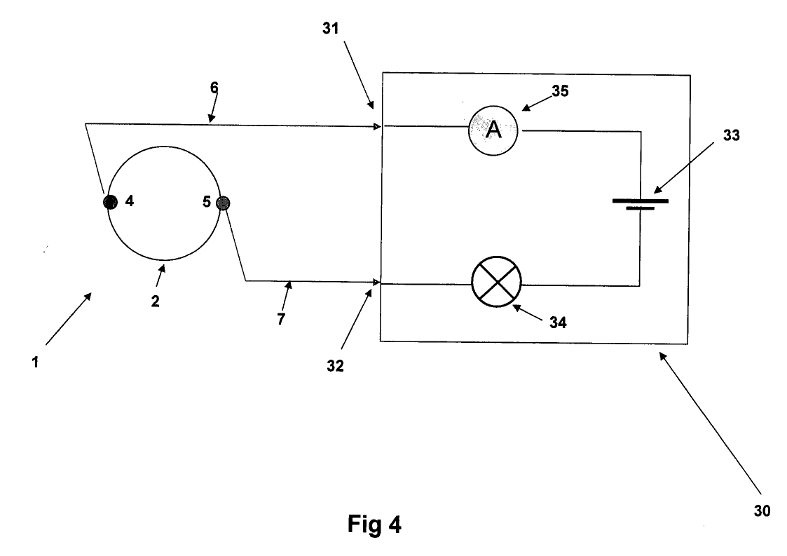

FIG. 4 shows a schematic presentation of a sensor connected to a measuring unit,

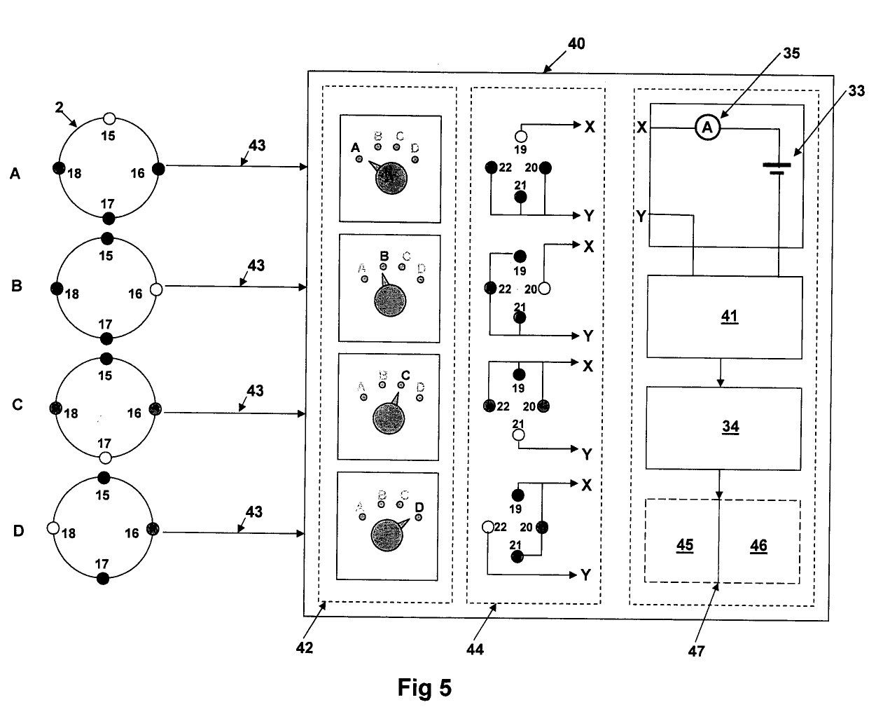

FIG. 5 shows a schematic presentation of the sensor of FIG. 3, an associated DC measuring unit and the switching modes for measurement conditions of the switching means during a measurement session

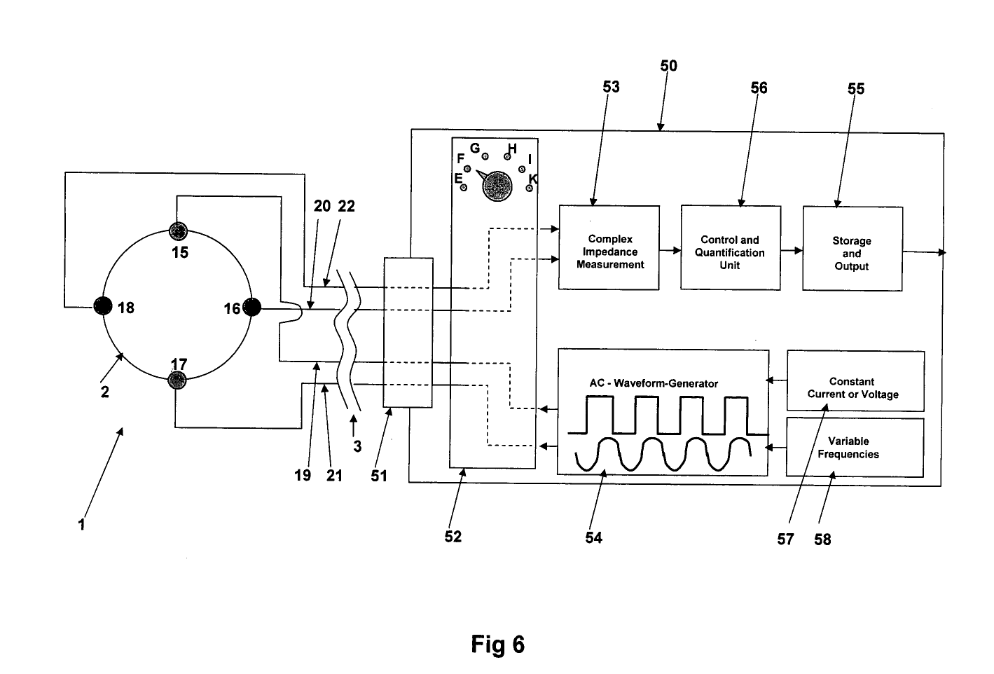

FIG. 6 shows a schematic presentation of the sensor of FIG. 3 and an associated AC measuring unit

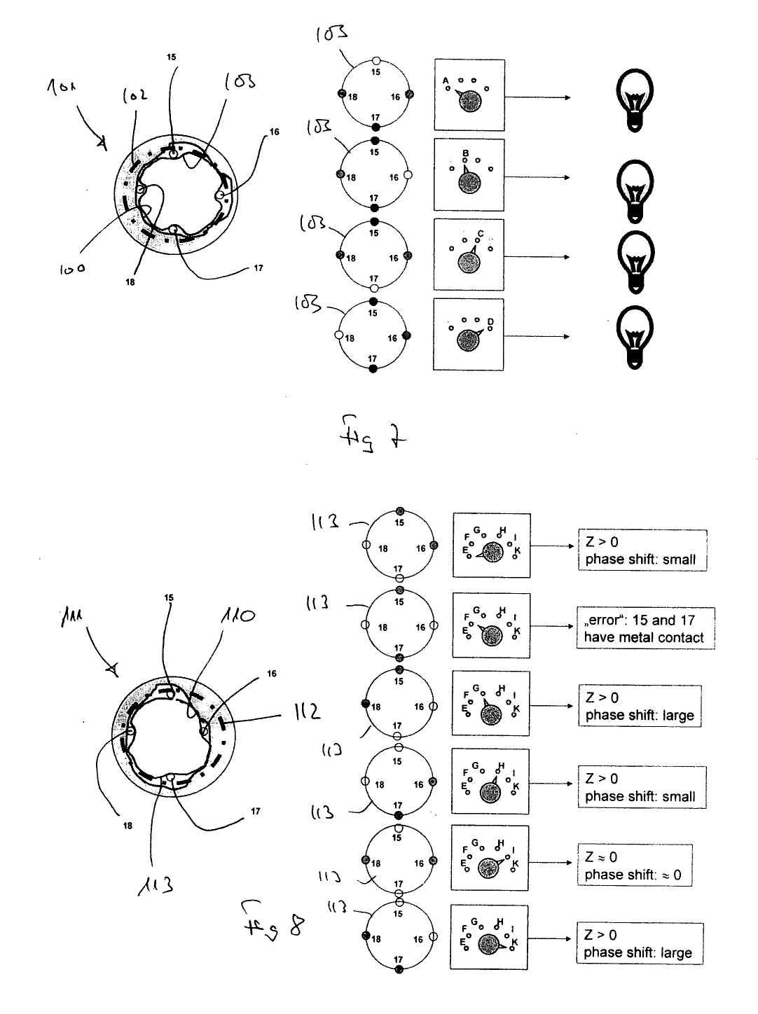

FIG. 7 shows an example of bipolar DC or AC measurement according to the invention

FIG. 8 shows an example of quadrupolar AC measurement according to the invention

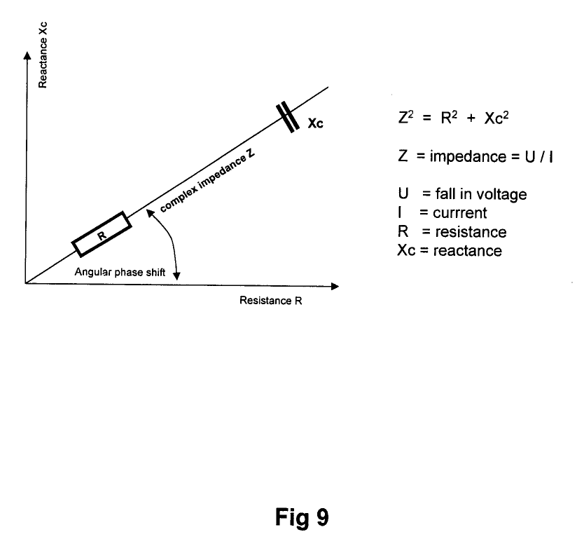

FIG. 9 shows a diagram of complex impedance measurement

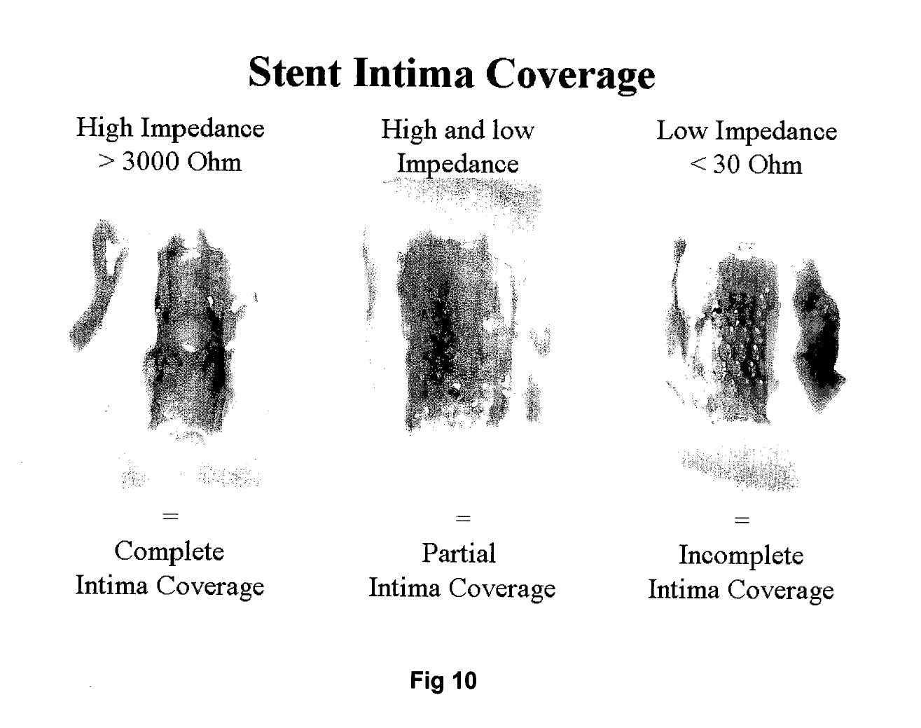

FIG. 10 shows pictures taken of stents being covered completely, partial or incomplete with intima.

FIGS. 1 and 2 show a sensor designed as endovascular catheter 1 with a sensing section designed as inflatable balloon 2 and means, in a preferred embodiment designed as a shaft 3, which are adapted to move the sensing section within a vessel of a body towards the position of a stent being implanted in the vessel and to move the sensing section operationally into this stent as known to the ones skilled. For the sake of simpliness, the means hare hereinafter denoted as shaft 3, although evidently every suitable means to bring the sensor into its operative position is included in the present invention. Balloon 2 and shaft 3 may completely be designed according to the state of the art. However, the balloon 2 supports or bears two electrodes 4 and 5, attached to the balloon and protruding over its surface such that electrode contact occurs with the inner surface of a stent, if the balloon 2 is introduced into a stent to examine its coverage with neointima. Leads 6,7 are wound around the shaft 3 and end up in a connector 8 adapted to be connected to a measuring unit. Of course, the leads 6,7 can be arranged on the shaft 3 in any suitable matter, e.g. with greater pitch than shown or in parallel to the shaft to avoid inductivity.

The figure shows further that the electrodes 4 and 5 are arranged such that the direction of a current flowing between associated electrodes is substantially transverse to the longitudinal axis of the sensing section. This is the case, when the sensing section or balloon 2 is introduced into a stent and the electrodes contact its inner surface due to the absence of intima coverage. Of course, if there is intima coverage, a current between the electrodes is not completely blocked, but there is remarkably enhanced resistance or impedance due to neointima layer.

An electrode configuration or a pattern of electrodes arranged such that a current flowing between associated electrodes is directed lengthwise is possible and within the spirit of the present invention, but would imply a need for a larger number of electrodes, as due to the length of the balloon 2 compared to its diameter, the distance between electrodes arranged lengthwise is smaller than between electrodes arranged in a transverse direction, such as e.g. electrode rings. A smaller distance is suitable, however, to sense accordingly smaller areas of irregular intima coverage.

Consequently, the electrodes extend in a direction substantially parallel to the axis and over substantially the length of the sensing section, with the advantage that a large area of the relevant inner stent surface is contacted with only two electrodes. This is also advantageous in terms of the connection of the electrodes with the measurement unit described below by means of e.g. leads, as a low number of leads facilitates the wiring needed along the shaft 3.

Preferably, at least one electrode is shaped as a filament, to guarantee smooth contact with the inner stent surface, being covered with intima or not. However, it is also possible to arrange laminar, and/or meander-shaped electrodes, to ensure a larger contact area of the inner stent surface, if such electrodes are enough bendable or flexible to contact the inner surface without gap in the case it is somewhat uneven, especially when covered with tissue like neointima. In a preferred embodiment, the laminar electrodes cover almost the full inner stent surface and are separated from each other by small filament like gaps. This allows to detect minor or very small surface areas of the inner stent surface not covered with neointima, provided that there are at least two of such areas present, contacted by different electrodes or that the gap between different electrodes lies right over such an area.

Alternatively, at least one of, preferably all of, the electrodes are laminary elongated. Then, larger areas of the inner surface 100,110 are contacted by one and the same electrode, with increased likelihood to also contact the spots without coverage of neointima.

Further alternatively, at least one of, preferably all of, the electrodes have a certaing height, i.e. are of three dimensional shape. Consequently, local pressure by the electrode surface is increased and the contact to neointima or to the stent improved. The one skilled will design such a shape of the electrodes according to the specific needs of measurement, considering e.g. the kind of implant to be investigated. A pattern of pimples, as well as a pattern of filaments with a triangle like cross section, or a pattern with mixed shaped electrodes etc. may be suitable for a specific use of a sensor designed accordingly.

In a further embodiment, the electrodes are made of a material that is visible to x-rays, such that the precise positioning of the sensor, as well as a possible shift or slip in the stent, can be checked by means of x-ray equipment.

The electrodes can be secured to the balloon 2 by means of two attachment rings 9,10. Therefore, if the balloon 2 is inflated, the electrodes may be stretched but will not break apart or will not detached from their original position. Bended portions 11, 12 ensure enough stretching possibilities for the electrodes, when the balloon is inflated.

FIG. 3 shows a preferred embodiment of the inventive sensor, whereby the sensing section is designed as balloon 2. Any expandable sensing section adapted to be introduced into a stent and supporting a pattern of electrodes circumferentially extending on its outer surface area is included in the present invention. Even a sensing section with non-expandable body is also included, if insertable into a stent, under the overall condition that the electrodes are arranged for operationally contacting the inner surface of the stent. The latter condition includes a smooth contact with minimum risk of damaging the vessel or intima structure.

FIG. 3 shows four electrodes 15,16,17,18 arranged symmetrically on the circumference of, and extending substantially over, the length of the sensing section. Furthermore, the electrodes are circumferentially spaced by 90-degree circular sectors. Consequently, there are four leads 19, 20, 21, 22 attached to the shaft 3 and leading to a connector 8 intended to be connected to a measuring unit such as the ones as described below.

The circumferentially extending pattern of electrodes, preferably designed symmetrically, ensures best possible detection of non-uniform coverage of tissue, such as neointima. Because of the uniform, net-like electrode contacts with the inner surface of a stent, even small areas of a non-endothelialized stent are detectable, when the electrodes are connected to a measuring unit as described below. Therefore, any electrode configuration or pattern is useful, if circumferentially extending on the outer surface area of the sensing section, and if connected or connectable to a measuring unit as described below. Even an arrangement of several electrodes helically wound around the sensing section or balloon 2, but preferably equally spaced (in the case of four electrodes by 90°) may be used. However, the configuration or pattern as shown in the Figures are preferred due to the minimal amount of leads 6,7 or 19,20,21,22 needed and the simple manufacturing.

A minimal amount of leads can be realized also, if the pattern of electrodes is wired groupwise on the sensing section such that only main wires or leads are needed to be attached to the shaft 3.

Impedance measurements within endovascular stents is based on the finding that stents with incomplete or missing neointimal coverage are associated with low-resistance (low-ohmic) values while stents with neointimal coverage comprise significantly higher resistance (high-ohmic) values.

Any endovascular device, such as wires devices or catheter devices, may provide transient mechanical (and electrical) contact of electrodes to the inner surface of the stented vessel segment and therefore can be used with the invention described herein.

For example, impedance measurements can be achieved using a balloon catheter 1 (e.g. over-the-wire or monorail) with integrated biocompatible electrical leads 4,5; 15,16,17,18 such as wires made of platinum-iridium or titanium. According to the number of electrodes, a bipolar balloon catheter (FIG. 1), a quadrupolar balloon catheter (FIG. 2), or a multipolar balloon catheter with more than four electrodes can be used. The wires 4,5; 15,16,17,18 at the distally located balloon 2 are electrodes (without insulation) which continue as leads 7,8; 19,20,21,22 (with insulation) at the catheter shaft 3 to the connector 8 at the proximal end of the catheter. The electrodes on the expandable platform (like the balloon 2) have preferably a circumferential-symmetric array, i.e., arrangement in identical circular sectors. For the production of microelectrodes, photomicrographic methods based on semiconductor technology may be used. The fixation of the microelectrodes 4,5; 15,16,17,18 to the balloon 2 is possible by using clamps 9, 10 at the catheter shaft, proximally and distally to the balloon 2. In addition, the electrodes can be integrated into very thin polyimide insulation which itself can be glued to the balloon 2 by using a biocompatible two component resin (11). This polyimide technology has been described previously (12). Each electrode 7,8; 19,20,21,22 may have bends to allow expansion and avoid rupture during balloon inflation. Synthetic coating of the leads 7,8; 19,20,21,22 at the catheter shaft 3, for example, with silicon or polyurethane, may help insulating and/or stabilizing the leads 7,8; 19,20,21,22 at the catheter shaft. 3 The connector 8 provides plug-in connections for separate, reusable measurement cables that connect to the external, reusable measurement unit as described below.

The balloon catheter 1 is inserted into the vessel segment containing the implanted stent for endovascular impedance measurements. The balloon is then being inflated with adequate pressure to ensure reliable contact of the balloon electrodes with the inner surface of the vessel wall containing neointima or the uncovered stent itself.

FIG. 2: Bipolar balloon catheter. Panel A: longitudinal view of the tip of the balloon catheter showing an inflated balloon 2 with two electrodes. Both electrodes have parallel alignment in axial direction of the catheter. Panel B: cross-section of the tip of the balloon catheter showing the two electrodes with circumferential-symmetric array so that both electrodes are separated from each other by 180 degrees. Panel C: longitudinal view of the catheter shaft. The two insulated leads can be integrated into the shaft by double-helical winding. Instead of a helical winding, the leads can also be incorporated into the shaft using a parallel arrangement in axial direction. At the proximal end of the catheter, the two leads end at the connector with two plug-ins for two measurement cables.

FIG. 3: Quadrupolar balloon catheter. Panel A: longitudinal view of the tip of the balloon catheter showing an inflated balloon with four electrodes. The four electrodes have parallel alignment in axial direction of the catheter. Panel B: cross-section of the tip of the balloon catheter showing a circumferential-symmetric electrode array, i.e. the electrodes are arranged in quarter-circles at 90, 180, 270, and 360 degrees. Panel C: longitudinal view of the catheter shaft showing a possible lead alignment by quadruble-helical winding of the leads. The leads can also be incorporated into the shaft using a parallel arrangement instead of helical winding. At the proximal end of the catheter, the four leads end at the connector with four plug-ins for a quadrupolar measurement cable.

FIG. 4 shows a measuring unit according to the present invention in a basic embodiment for standard measurement sessions, e.g. to be performed with simple equipment for gross diagnosis or severe lack of neointima only.

Endovascular stent impedance measurements can be performed with direct current (DC) or with alternating current (AC). Standard measurement is performed with DC.

The bipolar DC measurement unit 30 is schematically shown, as is also a balloon 2 of a catheter 1 with electrodes 4,5 (see FIG. 1) and leads 6,7. A connector 8 (FIG. 1) and connecting means of the measuring unit 30 accordingly designed to connect the conductors or leads 6,7 to the unit 30 are symbolized by the arrows 31,32 and designed according to the state of the art. A DC generator, designed as a battery or power supply unit 33 is connected to DC measuring means adapted to measure DC resistance, designed as an illuminating diode 34 and/or an amperemeter 35.

As the diode 34 illuminates in case of a short circuit (see below), it also performs as output means of the unit 30 to display the measurement result.

Bipolar DC measurement is used to measure the ohmic resistance according to the all-or-none-law. In case of metallic contact of the electrodes 4,5 with the inner stent surface, the impedance will be low due to short-circuit. In case of non-metallic contact (neointima), the impedance will be substantially higher. In case of short current, an illuminating diode 34 may be used to indicate metal contact with the stent. Electrolysis effects in the stent area from DC can be minimized by using short periods of current conduction. AC (instead of DC) can also be used by including a function generator.

Test results of bipolar impedance measurement:

| | |
|---|---|
| No neointimal coverage (i.e. no | Current conduction, low impedance, |
| coverage in both of the areas of | diode 34 illuminated |
| electrodes 4 and 5) | |
| Neointimal stent coverage (i.e. | No current conduction, high |
| coverage in at least one of the | impedance, diode 34 not |
| areas of electrodes 4 and 5) | illuminated |

DC impedance measurement is also possible with a sensor having a sensing section with more than two, i.e. three or four or even more electrodes, to get a more precise response regarding intima coverage.

FIG. 5 shows schematically a measuring unit 40 with a DC generator 33 and output means 34 designed as illuminating diode 34 or e.g. monitor or printer. Schematically shown are DC resistance measuring means 41 and further switching means 42 adapted to connect the DC generator 33 in a predetermined manner with the four conductors or leads 19,20,21,22 (FIG. 3) to establish measurement condition of a measurement session,

Shown are the four measurement conditions or switch settings A, B, C and D; whereby starting from a balloon 2 with electrodes 15,16,17,18 (FIG. 3) the leads 19,20,21,22 (FIG. 3), here symbolized by arrow 43 are operatively connected with the unit 40 through coupling means not shown in FIG. 5.

The switching means establish the four different measurement conditions A to D according to the diagrammatic illustrations 44 symbolizing the specific switching status of measurement condition A to D in FIG. 5. This illustration again symbolizes the conductors 19 to 21 of the shaft 3 (FIG. 3), and their connection with the DC generator 33 via the input X and Y.

Measurement condition A shows that individual conductor 19 is connected to the DC generator 33, while conductors 20,21,22 are connected in parallel and also connected with DC generator 33.

Measurement condition B shows that individual conductor 20 is connected to the DC generator 33, while conductors 19,21,22 are connected in parallel and also connected with DC generator 33.

Measurement condition C shows that individual conductor 21 is connected to the DC generator 33, while conductors 19,20,22 are connected in parallel and also connected with DC generator 33.

Measurement condition D shows that individual conductor 22 is connected to the DC generator 33, while conductors 19,20,21, are connected in parallel and also connected with DC generator 33.

These four measurement conditions A to D are part of a predetermined procedure to perform resistance measurement over various electrode combinations for a measurement session including all the necessary measurements to determine coverage of tissue as neointima on a stent surface.

Storage means 45 for storing measurement results and a control unit 46 to control proper execution of the measurement session and proper display of the result are symbolized by the dotted box 47.

In summary, the predetermined procedure includes measuring DC resistance between a first individual conductor and all of the other conductors being connected in parallel, and to repeat such measurement with a further individual conductor and all of the other conductors also being connected in parallel, and to repeat such measurement until the resistance between each of the conductors and the in each case remaining conductors has been measured individually at least once.

By performing the full procedure for a measurement session, any area of irregular neointima coverage extending over at least two electrodes 15 to 18 will be detected. Furthermore, by increasing the amount of electrodes even smaller areas are detectable, because the distance between adjacent electrodes declines.

In a further embodiment, instead of a DC generator, an AC generator may be used. Then, possible disadvantageous effects of DC used on the living body can be avoided. By doing so, the basic construction of unit 40 remains unchanged.

The hardware construction of the unit 40 with an AC or with a DC generator 33, as shown in FIG. 5 can easily be detailed and built by the one skilled. In particular, the one skilled can built the adequate electronical equipment to provide for a fully automatical unit 40, even with an interface in addition or instead of the storage and output means 55; 45,46 to send the information about stent coverage to further equipment.

Quadrupolar DC impedance measurement enables a principle of rotational impedance measurement. The rationale of using rotational impedance measurement for detecting neointimal stent coverage is the possibility that bipolar measurement (FIG. 4) may not be sensitive enough for detecting partially uncovered stent struts, because for such detection, different electrodes have to get contact with uncovered areas of inner stent surface. For example, if the first electrode 4 (FIG. 1) has metal contact from an uncovered stent strut but the second electrode 5 (FIG. 1) has no metal contact, there would be no current conduction and a high impedance value is measured. In other words, bipolar impedance measurement may not differentiate between complete and partial neointimal stent coverage. Quadrupolar rotational impedance measurement as shown in FIG. 5 is more sensitive than bipolar measurement because of the following reason: With each measurement one of the electrodes 15 or 16 or 17 or 18 is electrically separated and tested against the other three electrodes 16,17,18 or 15,17,18 or 15,16,18 or 15,16,17 which are electrically interconnected by switching means 42 (FIG. 5). These four switch settings or measurement conditions are shown in FIG. 5.

In the other switch settings, i.e., measurement conditions, the next electrode is then electrically separated and tested against the residual electrodes so that all four electrodes are measured once. Therefore, quadrupolar rotational DC impedance measurement differentiates between missing, partial, or complete neointimal stent coverage.

Test results of quadrupolar DC impedance measurement:

| | |
|---|---|
| Complete neointimal stent | No current conduction in all four switch |
| coverage | settings |
| Partial neointimal stent | Current conduction in at least one, but not |
| coverage | all of the A to D switch settings only |
| No neointimal stent coverage | Current conduction in all four switch |
| | settings |

As mentioned above, more than four electrodes can be used in the same manner to get a more detailed picture of intima coverage of the inner stent surface.

AC (instead of DC) can also be used by including a function generator. If so, and as explained with regard to FIG. 8, the reactance xc allows to reason about properties of the tissue covering inner stent surface.

FIG. 6 shows AC measurement according to a further preferred embodiment.

A balloon 2 of a catheter 1, supporting electrodes 15 to 18 (FIG. 3) is operatively connected to a measurement unit 50 by means of connectors 19 to 22 (FIG. 3) attached to a shaft 3 (not shown in the figure) and connected to the unit 50 by connector 8 (FIG. 3), which is inserted in coupling means of the unit 50 as symbolized by box 51. Switching means 52 connect two of the connectors 19 to 22, i.e. connectors 19 and 21 with the AC generator 54 and the other two connectors 20 and 22 with a complex impedance detecting arrangement 53.

Storage means and output means are symbolized by box 55.

A control unit 56 is in control of the procedure of the measurement session carried out by unit 50.

The AC generator 54 is adapted to generate during a measurement session constant current and/or constant voltage 57 of different preselected frequencies 58, and is also adapted to generate during a measurement session preselected waveforms, preferably sinusoidal and/or rectangular waves.

The complex impedance detecting arrangement 53 is adapted e.g. for detecting the angular phase shift between voltage and current and the real part of the impedance during a measurement condition.

The storage means 55 are adapted to store measurement results and all intermediate data needed to carry out the measurement session.

The output means 55 are adapted to transmit and/or display measurement results of the measuring session including the values of the real part of the impedance measured and the corresponding values of the angular phase shift measured.

Transmittal includes copying of data to an other electronic device; displaying includes generating a printout or displaying the data on a screen.

The complex impedance detecting arrangement 53 being adapted for detecting the angular phase shift between voltage and current and the real part of the impedance during a measurement condition, and the output means further being adapted to transmit and/or display measurement results of the measuring session including the values of the real part of the impedances measured and the corresponding values of the angular phase shifts measured.

The examination of the phase interface of metal-neointima is conceived with electrical methods, such as current-voltage measurements, current-time measurements, or voltage-time measurements. The preset variables current or voltage can be kept constant 57 (steady-state measurement methods—application of a constant-current source or constant-voltage source) or could be modified as a function of time 54 (unsteady measurement methods—application of a function generator). Commonly used unsteady methods comprise linear, stepwise, rectangular, or sinusoid modification of the preset variable.

The rationale for quadrupolar AC impedance measurement of stents includes the following: Constant current is provided from a power source through a resistor of unknown resistance. The fall of voltage is then measured at the site of the resistor. Current source and voltmeter are integrated in the measurement unit 53. If only two leads are used to connect to the resistor, the measured impedance will inevitably include the innate impedance of these leads. The innate lead impedance cannot simply be subtracted from the measurement result, because the contact impedance between the balloon electrodes and the stent may vary, depending on the inflation pressure of the balloon. With quadrupolar AC impedance measurement, two electrodes are used as power supply and two separate electrodes are used as sensors for measurement of voltage. Here, the measurement results are independent from innate lead impedance and contact impedance values, because 1) preset current is supplied by a constant current source independent from the present impedance, and 2) no current conduction into the voltmeter occurs during voltage measurement (i.e., an ideal voltmeter with infinitly large input resistance). Without current conduction there is no fall of voltage, and the resistance of the sensor leads is negligible. As a result, an unaltered measurement of voltage and thus reliable measurement of impedance can be achieved. Therefore, contemporary measurement systems preferable use quadrupolar AC measurement units (four-pole technique) (FIG. 6).

Quadrupolar AC measurement systems are based on the complex impedance, consisting of the real and imaginary part of the AC measurement. Importantly, the impedance depends on the AC frequency. A homogeneous electrical field is applied via two electrodes number 15 and 17 with constant current and high frequency. The electrodes number 16 and 18 are used as sensor electrodes. This approach guarantees galvanic isolation and precludes adverse bias effects.

The ohmic resistance (R) that is measured with low frequencies represents the real part and mostly depends on the resistance of plasma fluids and electrolytes. The resistance (reactance Xc) that is measured with high frequencies represents the imaginary part and mostly depends on the capacitive properties of cell membranes of the neointimal and endothelial cells. Thus, the imaginary part of the complex impedance measurement Xc represents a measure of the neointimal thickness within a metal stent. The ratio of reactance and resistance is preferably expressed by the angular phase shift which is a measure of the phase difference in voltage and current at the sensor electrodes number 2 and 4 (see FIG. 7).

As already described in FIG. 5, the switching means 52 can alter the switch settings or measurement conditions in a predetermined manner or according to a predetermined procedure, respectively.

In this embodiment, six measurement conditions E, F, G, H, I and K combine to a measurement session for an effective and sufficient detection of the tissue coverage of the inner surface of a stent.

The six measurement conditions or switch settings E to K as shown in FIG. 6 (box 52) are summarized below and include the following electrode combinations:

| | Current conducting | |
|---|---|---|
| measurement condition | electrodes | Sensing electrodes |
| E | 15 and 16 | 17 and 18 |
| F | 15 and 17 | 16 and 18 |
| G | 15 and 18 | 16 and 17 |
| H | 16 and 17 | 15 and 18 |
| I | 16 and 18 | 15 and 17 |
| K | 17 and 18 | 15 and 16 |

In other words, the switching means 52 are adapted to connect two of the conductors 19 to 22 with the AC generator 54 and the remaining conductors with the complex impedance detecting arrangement 53 to establish measurement condition, the switching means 52 are further adapted for altering the measurement condition during the measurement session according to a predetermined procedure (see the table above), thereby performing impedance measurement over various electrode combinations to ensure effective and sufficient detection of tissue or lack of tissue on the inner surface of a stent.

The predetermined procedure includes to consecutively connect any of the connectors 19 to 22 with another one of the connectors with the AC generator 54 and the remaining connectors with the complex impedance detecting arrangement 53 such that all of the possible pairing combinations of the conductors 19 to 22 connected to the AC generator 54 have operatively been performed once.

For each measurement condition A to K, the AC complex impedance detecting arrangement 53 detects the angular phase shift between voltage and current and the real part of the impedance for the following purpose:

Similar to the rotational DC impedance measurement, quadrupolar AC impedance measurement can differentiate between missing, thin, or tick neointimal stent coverage.

Test results of quadrupolar AC impedance measurement include for each of the measurement conditions E to K:

| | |
|---|---|
| Thick neointimal stent | Real part of impedance > 0 |
| coverage | Angular phase shift: large |
| Thin neointimal stent coverage | Real part of impedance > 0 |
| | Angular phase shift: small |
| No neointimal stent coverage | Real part of impedance ≈ 0 |
| | Angular phase shift ≈ 0 |

For purposes of completeness only shows FIG. 9 a diagram with the relationship between Reactance Xc and Resistance R, as it is know to the one skilled.

The pictures of FIG. 10 resulting from vivo testing as described below confirmed the test results according to the table above.

The sophisticated options with quadrupolar AC measurements allow to investigate the complex impedance of the neointimal stent healing process.

Finally, the output means are adapted to transmit and/or display measurement results of the measuring session including the values of the real part of the impedances measured and the corresponding values of the angular phase shifts measured.

FIG. 7 shows an example of DC measurement of the inner surface 100 of a vessel 101 in a cross sectional view, whereby a stent 102 inserted into the vessel 101 is shown. A balloon 103 with electrodes 15 to 18 is inserted into stent 102 and inflated, such that the electrodes 15 to 18 are pressed against the inner surface 100 or the stent 102 respectively. For the purposes of FIG. 3, there is no distinction made between neointima and other tissues of the vessel 101, furthermore, a shaft of the catheter bearing balloon 103 is not shown.

As can be seen from FIG. 7, electrodes 15 and 16 are in contact with stent 102, while electrodes 17 and 18 are pressed against tissue (neointima) of vessel 101, covering stent 102 in this area.

As also can be seen from FIG. 7, in the middle section, electrodes 15 to 18 are now connected to realize measurement conditions A to D as described above in connection with FIG. 5. Measurement condition A connects in parallel electrodes 16 to 18, therefore current is flowing from electrode 15 through stent 102 to electrode 16. The burning light shows electrical contact, i.e. close to zero resistance.

Once all the measurement conditions A to D have been made, it is clear that electrodes 15 and 16 are in electrical contact with stent 102, and electrodes 17 and 18 are not.

FIG. 8 shows an example of quadropolar AC measurement of the inner surface 110 of a vessel 111 in a cross-sectional view, whereby a stent 112 inserted into the vessel 111 is shown. A balloon 113 with electrodes 15 to 18 is inserted into stent 112 and inflated, such that the electrodes 15 to 18 are pressed against the inner surface 110 or the stent 112 respectively. For the purposes of FIG. 8, there is no distinction made between neointima and other tissues of the vessel 111, furthermore, a shaft of the catheter bearing balloon 113 is not shown.

As can be seen from FIG. 8, electrodes 15 and 17 are in contact with stent 112, while electrodes 16 and 18 are pressed against tissue (neointima) of vessel 111, covering stent 102 in this area.

As AC measurement is done, there are different electrode connections compared to those of FIG. 7; the electrode connections of FIG. 8 are depicted with E to K and described in the table above. Current conducting electrodes are shown filled, and impedance sensing electrodes are shown as empty circles.

First, if current conducting electrodes are in contact with the stent 112, there is a short circuit between them, such that no current can be sensed by the sensing electrodes, which can be shown as “error” message (see measurement condition F in FIG. 8).

Then, if there is no short circuit between the current conducting electrodes, an impedance will be measured between the sensing electrodes, except in the case that both of the sensing electrodes contact stent 112, such that the impedance Z (ohmic resistance as well as the phase shift) are close to zero. See measurement condition I of FIG. 8.

In the remaining cases, either one or both of the sensing electrodes do not contact the stent 112, but the inner surface 110 of the vessel 111, i.e. neointima. Therefore, the real part of the impedance Z is greater than zero, and there is an imaginary part of impedance Z, i.e. a phase shift between voltage and current (see FIG. 9 due to the capacitive properties of neointima.

Contact of one of the electrodes with stent 112 and contact of the inner surface 111 by the other electrode, whereby the thickness of the layer of neointima is low causes a small phase shift, see measurement condition E and H as well of FIG. 8.

In measurement condition G and K at least one of the sensing electrodes is pressed against a more thick layer of neointima, consequently the phase shift is large.

It goes without saying that the one skilled is in a position to determine an adequate number of electrodes and the adequate measurement conditions as well, to generate the desired information of neointima coverage of a stent.

In this respect, reference is made to the description of FIG. 3 regarding a groupwise wiring of the electrodes on the sensing section. In particular, but not limited to the following example, in the case that rings are used as electrodes, a pattern of parallel rings can be arranged over the length of the sensing section, such as a ballon 2. Then, preselected rings are made associated rings, and therefore connected to different conductors, such that the measurement conditions as described above can be established. Now, as an example, the first four rings are connected to four conductors, which in turn are further connected to the measuring unit. Then the second group of the next four rings are connected to only a corresponding ring, i.e. the fifth ring to the first one, the sixth ring to the second one, the seventh ring to the third one, and the eight ring to the fourth one. A possible third (or further) group of four rings will be connected in the analogous way. At the end, the first, fifth, ninth etc ring are connected with the first conductor, the second, sixth and tenth ring are connected with the second conductor etc. By doing so, each of the areas covered by one group of four rings is measured in parallel, and the number of electrodes can be enhanced without restrictions given by the arrangement of a corresponding number of conductors on shaft 3. Consequently, the electrodes may be arranged as close and as numerous as desired by the one skilled for specific detection purposes.

In a summary, at least one preselected group of electrodes are operatively interconnected to each other by a common conductor, for further connection of this at least one group of electrodes to a measuring unit.

Therefore, it is well understood that both, DC and AC measurement can be performed with sensing sections having a different number of electrodes, from two electrodes to a number exceeding four electrodes according to the desired detecting result: the more electrodes are being used, the smaller the possibility to miss an uncovered area on the inner surface of a stent. The preferred embodiments as described are not intended to limit the number or arrangement of the electrodes for the purposes of the present invention.

Accordingly, the measurement unit can be equipped to perform measurement sessions with catheters having two or more electrodes as described above, by adopting the coupling means, the adapting means or the switching means, the DC resistance measuring means or the AC complex impedance measuring means, the storing means, the output means and finally the control means by the one skilled in the way described above.

Tests were done as follows:

A) In-Vitro Testing

In a first step, it was shown that commonly used coronary stents have low impedance values. The impedance of the following coronary stents (3.0 mm in diameter) was measured by applying direct current (3.0 Volt) to the ends of the metal stent. Measurements were performed using a conventional impedance measurement device (Kopp Instruments GMT-19A).

Cypher® Stent: 4 Ohm

Taxus® Stent: 4 Ohm

Promus® Stent: 6 Ohm

Prokinetic® Stent: 5 Ohm

In a second step, impedance measurements of the above mentioned stents were performed using a bipolar, 3.0×20 mm, over-the-wire balloon catheter prototype (FIG. 1).

The following impedance values were obtained by inserting the balloon into the stent and inflating the balloon with 8 atmospheres to achieve adequate mechanical contact of the electrodes to the stent. The displayed measurement results are the sum of both, the innate resistance of the stent and the conductor resistance of the catheter prototype:

Cypher® Stent: 15 Ohm

Taxus® Stent: 15 Ohm

Promous® Stent: 17 Ohm

Prokinetic® Stent: 15 Ohm

B) In-Vivo Testing

In each of 4 pigs, one 3.0×18 mm coronary metal stent was implanted into the left anterior descending artery, one stent into the left circumflex artery, and one stent into the right coronary artery. The stents were implanted with 16 atmospheres and 10 seconds balloon inflation time.

Six weeks after implantation, the pigs were euthanized and hearts were fixated in formalin. After fixation, 11 cuboid myocardial blocks containing the coronary artery segment with the implanted stent were obtained for impedance measurements.

The 3.0×20 mm balloon catheter prototype was directly inserted without a guide wire into each of the 11 myocardial blocks containing the coronary artery segment with the implanted stent. The balloon was then inflated with 8 atmospheres using a conventional indeflator once the balloon was completely inside the stent. Three impedance values were obtained from each stent segment by rotating the bipolar balloon catheter by approximately 45 degrees with each measurement. Three groups could be formed based on the type of measurements (Table):

1. Group: 7 stents showed consistently high impedance values

2. Group: 2 stents showed high and low impedance values

3. Group: 2 stents showed consistently low impedance values

All 7 stents with consistently high impedance values were macroscopically covered by thick neointima (FIG. 8, Panel A). Two stents had a mix of high and low impedance values and both showed areas of thin neointima stent coverage and areas of uncovered stent struts (FIG. 8, Panel B). Two stents had consistently low impedance values and both showed entirely missing neointimal coverage (FIG. 8, Panel C).

TABLE
Impedance values in coronary stents

| | Intima Coverage by Impedance | | |
|---|---|---|---|
| | Consistently | Mix of high | Consistently |
| Impedance (Ohm) measurements | high | and low | low |
| Number of stents | 7 | 2 | 2 |
| Number of measurements per stent | 3 | 3 | 3 |
| Number of total measurements | 21 | 6 | 6 |
| Impedance, mean ± SD, Ohm | 5910 ± 1583* | 2426 ± 2643* | 16 ± 4* |
| Impedance, median, Ohm | 6000 | 2261 | 15 |
| Impedance, range, Ohm | 3000-8500 | 12-5000 | 12-20 |
| Number of measurements ≧ 30 Ohm, % | 21 (100) | 3 (50) | 0 (0) |
| Number of measurements < 30 Ohm, % | 0 (0) | 3 (50) | 6 (100) |

*unpaired t-test: p = 0.004 between first and second group; p < 0.001 between first and third group; p = 0.049 between second and third group.

### REFERENCES

1. Daemen J, Wenaweser P, Tsuchida K, et al. Early and late coronary stent thrombosis of sirolimus-eluting and paclitaxel-eluting stents in routine clinical practice: data from a large two-institutional cohort study. Lancet 2007;369(9562):667-78.
2. Iakovou I, Schmidt T, Bonizzoni E, et al. Incidence, predictors, and outcome of thrombosis after successful implantation of drug-eluting stents. Jama 2005;293(17):2126-30.
3. Mauri L, Hsieh W H, Massaro J M, Ho K K, D'Agostino R, Cutlip D E. Stent thrombosis in randomized clinical trials of drug-eluting stents. N Engl J Med 2007;356(10):1020-9.
4. Lagerqvist B, James S K, Stenestrand U, Lindback J, Nilsson T, Wallentin L. Long-term outcomes with drug-eluting stents versus bare-metal stents in Sweden. N Engl J Med 2007;356(10):1009-19.
5. Matsumoto D, Shite J, Shinke. T, et al. Neointimal coverage of sirolimus-eluting stents at 6-month follow-up: evaluated by optical coherence tomography. Eur Heart J 2007;28(8):961-7.
6. Siqueira D A, Abizaid A A, Costa Jde R, et al. Late incomplete apposition after drug-eluting stent implantation: incidence and potential for adverse clinical outcomes. Eur Heart J 2007;28(11):1304-9.
7. Kotani J, Awata M, Nanto S, et al. Incomplete neointimal coverage of sirolimus-eluting stents: angioscopic findings. J Am Coll Cardiol 2006;47(10):2108-11.
8. Yusuf S, Zhao F, Mehta S R, Chrolavicius S, Tognoni G, Fox K K. Effects of clopidogrel in addition to aspirin in patients with acute coronary syndromes without ST-segment elevation. N Engl J Med 2001;345(7):494-502.
9. Steinhubl S R, Berger P B, Mann J T, 3rd, et al. Early and sustained dual oral antiplatelet therapy following percutaneous coronary intervention: a randomized controlled trial. Jama 2002;288(19):2411-20.
10. Bhatt D L, Fox K A, Hacke W, et al. Clopidogrel and aspirin versus aspirin alone for the prevention of atherothrombotic events. N Engl J Med 2006;354(16):1706-17.
11. Suselbeck T, Thielecke H, Weinschenk I, et al. In vivo intravascular electric impedance spectroscopy using a new catheter with integrated microelectrodes. Basic Res Cardiol 2005;100(1):28-34.
12. Rodriguez F J, Ceballos D, Schuttler M, et al. Polyimide cuff electrodes for peripheral nerve stimulation. J Neurosci Methods 2000;98(2):105-18.

1-30. (canceled)

31. Arrangement of a sensor having a sensing section adapted to be introduced into an implant, such as a stent and including a plurality of electrodes arranged in a pattern circumferentially extending on the outer surface area of the sensing section, and being further arranged for operationally contacting the inner surface of the implant such as a stent, and of a measuring unit adapted to measure DC resistance and/or AC impedance including a short circuit between preselected electrodes according to at least one measurement condition established during a measurement session, whereby the electrodes are connectable to the measuring unit, such that the at least one measuring condition or the several measurement conditions during a measurement session can be consecutively established.

32. Arrangement according to claim 31, whereby the connection to the measuring unit is made by conductors, preferably wires.

33. Arrangement according to claim 32, whereby the connection between at least one preselected group of electrodes of the sensor and the measuring unit is made by a conductor common to this at least one group of electrodes.

34. Sensor to detect the intima coverage of an implant, such as a stent with a sensing section, adapted to be introduced into an implant such as a stent and including electrodes arranged on the outer surface area of the sensing section, and means adapted to move the sensing section within a vessel of a body towards the position of an implant such as a stent being implanted in the vessel and to move the sensing section operationally into this implant such as a stent, the means being further adapted to accommodate conductors to connect the electrodes operationally with a measuring unit, characterized in that the electrodes are arranged for contacting the inner surface of the implant such as a stent, whereby the electrodes are further arranged such that in case of operational contact with the inner surface, the direction of a current flowing between associated electrodes is substantially transverse to the longitudinal axis of the sensing section.

35. Sensor to detect the intima coverage of an implant such as a stent with a sensing section, adapted to be introduced into a stent and including electrodes arranged on the outer surface area of the sensing section, and having means adapted to move the sensing section within a vessel of a body towards the position of a stent being implanted in the vessel and to move the sensing section operationally into this stent, the means being further adapted to accommodate conductors to connect the electrodes operationally with a measuring unit, characterized in that the electrodes are arranged for contacting the inner surface of the implant such as a stent, whereby the sensing section comprises at least one electrode extending in a direction substantially parallel to the axis and over substantially the length of the sensing section.

36. Sensor according to claim 35, whereby the at least one electrode is shaped as a filament.

37. Sensor according to claim 35, whereby the sensing section includes at least two, preferably four electrodes, arranged symmetrically on the circumference of, and extending substantially over, the length of the sensing section.

38. Sensor according to claim 37, whereby the symmetrically arranged electrodes are further arranged equidistantially, the preferably four electrodes being circumferentially spaced by 90 degrees.

39. Sensor according to claim 35, whereby at least one of, preferably all of, the electrodes are laminary elongated.

40. Sensor according to claim 35, whereby the sensing section comprises an inflatable balloon, adapted to support the electrodes.

41. Sensor according to claim 35, whereby at least one preselected group of electrodes are operatively interconnected to each other by a common conductor, for further connection of this at least one group of electrodes to a measuring unit.

42. Measuring unit comprising coupling means to electrically connect the two conductors from a sensor to detect the intima coverage of an implant such as a stent, a DC generator, means adapted to connect the DC generator with the two conductors to establish measurement condition of a measurement session, and DC resistance measuring means adapted to measure DC resistance between conductors connected to the DC generator, and output means for transmitting and/or displaying measurement results.

43. Measuring unit comprising coupling means to electrically connect the at least three conductors from of a sensor to detect the intima coverage of an implant, a DC generator, switching means adapted to connect the DC generator in a predetermined manner with the at least three conductors to establish measurement condition of a measurement session, and DC resistance measuring means adapted to measure DC resistance between conductors connected to the DC generator according to the measurement condition as established, and further being adapted for altering the measurement condition during the measurement session according to a predetermined procedure to perform resistance measurement over various electrode combinations, storage means for storing measurement results, output means for transmitting and/or displaying measurement results, and a control unit adapted to control the procedure of a measurement session.

44. Measuring unit according to claim 43, whereby the predetermined procedure includes measuring DC resistance between a first individual conductor and all of the other conductors being connected in parallel, and to repeat such measurement with a further individual conductor and all of the other conductors also being connected in parallel, and to repeat such measurement until the resistance between each of the conductors and the in each case remaining conductors has been measured individually at least once.

45. Measuring unit according to claim 43, the switching means being adapted to connect the four conductors with the DC generator and the predetermined procedure includes measuring DC resistance between an individual conductor and the remaining conductors connected in parallel such that each of the four connectors is measured as individual connector once.

46. Measuring unit comprising coupling means to electrically connect the two conductors from a sensor to detect the intima coverage of an implant, an AC generator, means adapted to connect the AC generator with the two conductors to establish measurement condition of a measurement session, and AC complex impedance measuring means adapted to measure AC impedance between the two conductors connected to the AC generator, and output means for transmitting and/or displaying measurement results.

47. Measuring unit comprising coupling means to electrically connect the at least three conductors on from a sensor to detect the intima coverage of an implant, an AC generator, switching means adapted to connect the AC generator in a predetermined manner with the at least three conductors to establish measurement condition of a measurement session, and AC complex impedance measuring means adapted to measure complex AC impedance between conductors connected to the AC generator according to the measurement condition as established, and further being adapted for altering the measurement condition during the measurement session according to a predetermined procedure to perform complex impedance measurement over various electrode combinations, storage means for storing measurement results, output means for transmitting and/or displaying measurement results, and a control unit adapted to control the procedure of a measurement session.

48. Measuring unit according to claim 47, whereby the predetermined procedure includes measuring AC impedance between a first individual conductor and all of the other conductors being connected in parallel, and to repeat such measurement with a further individual conductor and all of the other conductors also being connected in parallel, and to repeat such measurement until the resistance between each of the conductors and the in each case remaining conductors has been measured individually at least once.

49. Measuring unit comprising coupling means to electrically connect the unit with the at least four conductorson a shaft of a sensor to detect the intima coverage of an implant, an AC generator, a complex impedance detecting arrangement, switching means adapted to connect two of the conductors with the AC generator and the remaining conductorswith the complex impedance detecting arrangement to establish measurement condition, the switching means being further adapted for altering the measurement condition during the measurement session according to a predetermined procedure to perform impedance measurement over various electrode combinations, storage means for storing measurement results, output means for transmitting and/or displaying measurement results, and a control unit adapted to control the procedure of a measurement session.

50. Measuring unit according to claim 49, whereby the predetermined procedure includes to consecutively connect any of the connectors with another one of the connectors with the AC generator and the in each case remaining connectors with the complex impedance detecting arrangement such that all of the possible pairing combinations of the conductors connected to the AC generator have operatively been performed once.

51. Measuring unit according to claim 49, whereby the AC generator is adapted to generate during a measurement session constant current and/or constant voltage of different preselected frequencies.

52. Measuring unit according to claim 49, whereby the AC generator is adapted to generate during a measurement session preselected waveforms, preferably sinusoidal and/or rectangular waves.

53. Measuring unit according to claim 49, the complex impedance detecting arrangement being adapted for detecting the angular phase shift between voltage and current and the real part of the impedance during a measurement condition, and the output means further being adapted to transmit and/or display measurement results of the measuring session including the values of the real part of the impedances measured and the corresponding values of the angular phase shifts measured.

54. Method to determine neointima coverage on the inner surface of an implant, such as a stent, characterized in that a sensing section having a pattern of electrodes arranged on its outer surface is introduced into the implant such as a stent such that the electrodes are in contact with the inner surface of the implant such as an implant such as a stent, and impedance measurements between selected electrodes are performed, whereby short circuits are distinguished from enhanced impedance between these electrodes and short circuits are interpreted as lack of intima coverage in the area of these electrodes.

55. Method according to claim 54, whereby increased angular phase shift in the impedance measured is interpreted as increased thickness of intima coverage in the area of these electrodes.

56. Method according to claim 54, whereby the pattern of electrodes is arranged such to evenly extend over the surface of the sensing section and selection of particular electrodes for an impedance measurement is done such that various combinations of electrodes for a different measurement have occurred at least once.

57. Use of a Sensor having a sensing section adapted to be introduced into an implant, such as a stent and including a plurality of electrodes arranged in a pattern circumferentially extending on the outer surface area of the sensing section, and being further arranged for operationally contacting the inner surface of the implant, such as an implant such as an implant such as a stent characterized in that the electrodes are connected to a measuring unit, adapted to measure DC resistance and/or AC impedance between preselected electrodes according to a measurement condition established during a measurement session, whereby the measurement condition is altered during a measurement session for consecutive measurements over various electrode combinations according to a preselected procedure, and in that measurement results with low DC resistance and/or low AC impedance with a small angular shift between voltage and current identify presence of areas of the inner surface of the an implant such as a stent with lacking coverage of neointima.

58. Use of a sensor according to claim 57, whereby the sensor further comprising a sensor to detect the intima coverage of an implant, such as a stent with a sensing section, adapted to be introduced into an implant such as a stent and including electrodes arranged on the outer surface area of the sensing section, and means adapted to move the sensing section within a vessel of a body towards the position of an implant such as a stent being implanted in the vessel and to move the sensing section operationally into this implant such as a stent, the means being further adapted to accommodate conductors to connect the electrodes operationally with a measuring unit, characterized in that the electrodes are arranged for contacting the inner surface of the implant such as a stent, whereby the electrodes are further arranged such that in case of operational contact with the inner surface, the direction of a current flowing between associated electrodes is substantially transverse to the longitudinal axis of the sensing section.

59. Use of a sensor according to claim 57, whereby the measuring unit further comprising measuring unit comprising coupling means to electrically connect the two conductors from a sensor to detect the intima coverage of an implant such as a stent, a DC generator, means adapted to connect the DC generator with the two conductors to establish measurement condition of a measurement session, and DC resistance measuring means adapted to measure DC resistance between conductors connected to the DC generator, and output means for transmitting and/or displaying measurement results.
